(19) United States
(12) Patent Application Publication
(10) Pub. No.: US 2026/0198995 A1
(43) Pub. Date: Jul. 16, 2026

(54) SURGICAL INSTRUMENTS AND SYSTEMS INCORPORATING ELECTROSURGICAL AND ULTRASONIC TREATING AND SENSING FUNCTIONALITY

(71) Applicant: Covidien LP, Mansfield, MA (US)

(72) Inventors: Matthew S. COWLEY, Frederick, CO (US); Richard L. CROFT, Mead, CO (US)

(21) Appl. No.: 19/564,700

(22) Filed: Mar. 12, 2026

Related U.S. Application Data
(63) Continuation of application No. PCT/IB2024/058920, filed on Sep. 13, 2024.
(60) Provisional application No. 63/583,063, filed on Sep. 15, 2023.

(51) Int. Cl. A61B 18/14; A61B 18/12; A61B 34/30; A61B 17/32; A61B 18/00

(52) U.S. Cl. CPC A61B 18/1445; A61B 18/1206; A61B 34/30; A61B 2017/320074; A61B 2017/320095; A61B 2018/00077; A61B 2018/0063; A61B 2018/00642; A61B 2018/00714; A61B 2018/00875; A61B 2018/00994; A61B 2018/1455

(57) ABSTRACT

A surgical instrument includes an end effector assembly having first and second jaw members configured to grasp tissue therebetween. The first jaw member including first and second electrically conductive tissue contacting plate portions configured to conduct Radio Frequency (RF) energy through tissue and the second jaw member including first and second ultrasonic tissue contacting plate portions configured to produce ultrasonic vibration energy and transmit the ultrasonic vibration energy to tissue. A surgical system includes an end effector assembly configured to grasp tissue and a surgical generator configured to output RF treatment energy, output an ultrasonic drive signal, sense RF energy feedback, sense ultrasonic energy feedback, and control the outputs based upon the feedback.

### CROSS-REFERENCE TO RELATED APPLICATIONS

This application is a continuation of International Patent Application No. PCT/IB2024/058920, filed September 13, 2024, which claims the benefit of U.S. Provisional Patent Application Serial No. 63/583,063, filed September 15, 2023, the entire contents of each of which applications are incorporated herein by reference. To the extent appropriate, a claim of priority is made to each of the above disclosed applications.

### FIELD

The present disclosure relates to energy-based surgical instruments and, more particularly, to surgical instruments and systems incorporating electrosurgical and ultrasonic treating and sensing functionality to facilitate energy-based tissue treatment.

### BACKGROUND

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue grasped between the electrodes or otherwise in contact therewith, to treat tissue. Alternatively, or additionally, an electrosurgical instrument or system may be configured to deliver monopolar RF energy from an active electrode to tissue in contact with the electrode, with the energy returning via a return electrode to complete the circuit.

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic end effector configured to vibrate at high frequencies, which allows for heating tissue to treat tissue grasped against or otherwise in contact with the ultrasonic end effector.

### SUMMARY

As used herein, the term “distal” refers to the portion that is being described which is farther from an operator (whether a human user (surgeon, nurse, etc.) or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with aspects of the disclosure is a surgical instrument including an end effector assembly having first and second jaw members. The first jaw member and/or the second jaw member is movable relative to the other between a spaced apart position and an approximated position for grasping tissue therebetween. The first jaw member includes first and second electrically conductive tissue contacting plate portions configured to conduct Radio Frequency (RF) energy through tissue grasped between the first and second jaw members. The second jaw member includes first and second ultrasonic tissue contacting plate portions configured to produce ultrasonic vibration energy and transmit the ultrasonic vibration energy to tissue grasped between the first and second jaw members.

In an aspect of the present disclosure, the first jaw member or the second jaw member further includes first and second walls extending from respective first and second outer peripheral sides thereof. The first and second walls include first and second electrically conductive surfaces, respectively. The first and second electrically conductive surfaces are electrically isolated from the first and second electrically conductive tissue contacting plate portions and configured to be energized to different potentials compared to the first and second electrically conductive tissue contacting plate portions to conduct RF energy therebetween and through tissue grasped between the first and second jaw members.

In another aspect of the present disclosure, the second jaw member further includes third and fourth electrically conductive tissue contacting plate portions. At least two of the first, second, third, or fourth electrically conductive tissue contacting plate portions are electrically isolated and configured to be energized to different potentials to conduct RF energy therebetween and through tissue grasped between the first and second jaw members.

In another aspect of the present disclosure, the third electrically conductive tissue contacting plate portion and the first ultrasonic tissue contacting plate portion are stacked relative to one another on the second jaw member, and the fourth electrically conductive tissue contacting plate portion and the second ultrasonic tissue contacting plate portion are stacked relative to one another on the second jaw member.

In still another aspect of the present disclosure, the first jaw member further includes third and fourth ultrasonic tissue contacting plate portions configured to produce ultrasonic vibration energy and transmit the ultrasonic vibration energy to tissue grasped between the first and second jaw members.

In yet another aspect of the present disclosure, the first electrically conductive tissue contacting plate portion and the third ultrasonic tissue contacting plate portion are stacked relative to one another on the first jaw member, and the second electrically conductive tissue contacting plate portion and the fourth ultrasonic tissue contacting plate portion are stacked relative to one another on the first jaw member.

In still yet another aspect of the present disclosure, the first and second ultrasonic tissue contacting plate portions are configured to sense at least one parameter of tissue in contact therewith.

In another aspect of the present disclosure, the first jaw member or the second jaw member includes an ultrasonic blade disposed between the plate portions thereof and configured to produce ultrasonic vibration energy for transmission to tissue grasped between the first and second jaw members. The ultrasonic blade may be formed from a piezoelectric material or may include a piezoelectric base and a transmission body coupled to the piezoelectric base.

In an aspect of the present disclosure, the ultrasonic blade is configured to sense at least one parameter of tissue in contact therewith.

In still another aspect of the present disclosure, the end effector assembly further includes a clevis. In such aspects, the first and second jaw members may be operably coupled to the clevis and movable relative to one another and the clevis between the spaced apart position and the approximated position.

In yet another aspect of the present disclosure, either or both of the first and second jaw members further includes a structural jaw frame supporting an insulative jaw body thereon. In such aspects the plate portions of the jaw member may be disposed on the insulative jaw body.

In another aspect of the present disclosure, the first and second electrically conductive tissue contacting plate portions are connected at ends thereof to define a U-shaped configuration. Alternatively, the first and second electrically conductive tissue contacting plate portions may be electrically isolated from one another.

In still yet another aspect of the present disclosure, the surgical instrument further includes a surgical robot having the end effector assembly releasably connectable thereto.

In another aspect of the present disclosure, the surgical instrument further includes a handle assembly operably coupled to the end effector assembly.

A surgical system provided in accordance with the present disclosure includes an end effector assembly including first and second jaw members. The first jaw member and/or the second jaw member are movable relative to one another between a spaced apart position and an approximated position for grasping tissue therebetween. The end effector assembly further including at least one electrically conductive tissue contacting plate portion (e.g., each disposed on one of the jaw members) and at least one ultrasonic tissue contacting plate portion (e.g., each disposed on one of the jaw members). The surgical system further includes a surgical generator configured to output Radio Frequency (RF) treatment energy to the at least one electrically conductive tissue contacting plate portion, output an ultrasonic drive signal to the at least one ultrasonic tissue contacting plate portion, sense feedback from the at least one electrically conductive tissue contacting plate portion, and sense feedback from the at least one ultrasonic tissue contacting plate portion. The surgical generator is configured to control the output of the RF treatment energy and the output of the ultrasonic drive signal to seal tissue grasped between the first and second jaw members based on the sensed feedback from the at least one electrically conductive tissue contacting plate portion and the sensed feedback from the at least one ultrasonic tissue contacting plate portion.

In an aspect of the present disclosure, the surgical generator is configured to determine a temperature of tissue grasped between the first and second jaw members, a stiffness of tissue grasped between the first and second jaw members, and/or a jaw pressure applied to tissue grasped between the first and second jaw members based on the sensed feedback from the at least one ultrasonic tissue contacting plate portion.

In another aspect of the present disclosure, the surgical generator is configured to determine an impedance of tissue grasped between the first and second jaw members based on the sensed feedback from the at least one electrically conductive tissue contacting plate portion.

In still another aspect of the present disclosure, the end effector assembly further includes an ultrasonic blade engaged to one of the first jaw member or the second jaw members. In such aspects, the surgical generator may be further configured to output an ultrasonic drive signal to the ultrasonic blade to dissect tissue grasped between the first and second jaw members.

In yet another aspect of the present disclosure, the surgical generator is configured to control the output using temperature-based control.

In still yet another aspect of the present disclosure, the surgical generator is configured to control the output to terminate the delivery of energy when the sensed feedback indicates that tissue grasped between the first and second jaw members is sealed.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 illustrates a surgical system provided in accordance with the present disclosure including a surgical instrument, a surgical generator and, in some aspects, a return electrode device;

FIG. 2 is a schematic illustration of a robotic surgical system provided in accordance with the present disclosure;

FIG. 3 is a side view of an end effector assembly provided in accordance with the present disclosure and configured for use with the surgical system of FIG. 1, the surgical system of FIG. 2, and/or any other suitable surgical system;

FIG. 4A is a transverse cross-sectional view of the end effector assembly of FIG. 3 with jaw members of the end effector assembly disposed in a spaced apart position;

FIG. 4B is a transverse cross-sectional view of the end effector assembly of FIG. 3 with the jaw members disposed in an approximated position;

FIGS. 5 and 6 are transverse cross-sectional views of other end effector assemblies provided in accordance with the present disclosure and configured for use with the surgical system of FIG. 1, the surgical system of FIG. 2, and/or any other suitable surgical system;

FIGS. 7 and 8 are transverse cross-sectional views of one of the jaw members of yet other end effector assemblies provided in accordance with the present disclosure and configured for use with the surgical system of FIG. 1, the surgical system of FIG. 2, and/or any other suitable surgical system; and

FIG. 9 is a block diagram of a surgical generator provided in accordance with the present disclosure and configured for use with the surgical system of FIG. 1, the surgical system of FIG. 2, and/or any other suitable surgical system.

### DETAILED DESCRIPTION

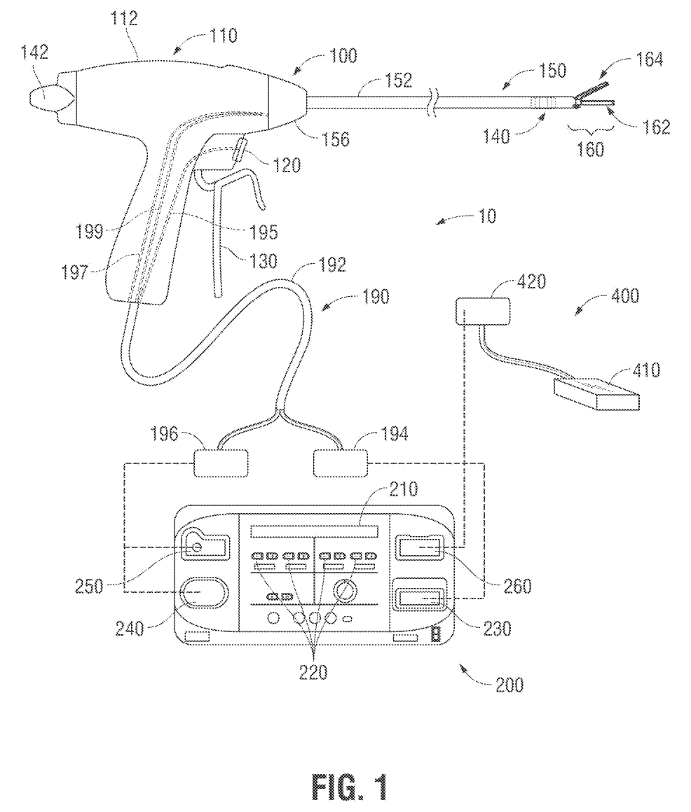

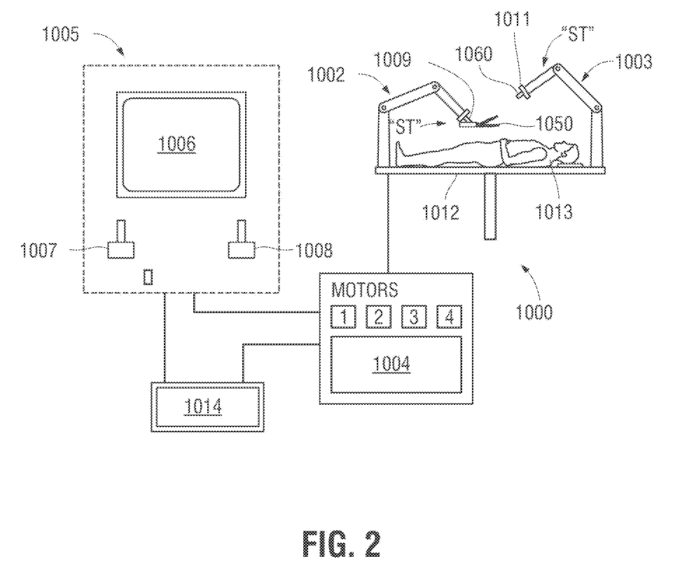

Referring to FIG. 1, a surgical system provided in accordance with aspects of the present disclosure is shown generally identified by reference numeral 10 including a surgical instrument 100, a surgical generator 200, and, in aspects, a return electrode device 400, e.g., including a return pad 410. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system such as, for example, robotic surgical system 1000 (FIG. 2) as detailed below.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. Surgical generator 200 is configured to produce ultrasonic drive signals for output through ultrasonic plug port 230 to surgical instrument 100 and/or to receive ultrasonic feedback signals from surgical instrument 100 through ultrasonic plug port 230 to enable one or more ultrasonic functions of surgical instrument 100. Surgical generator 200 is further configured to provide electrosurgical energy, e.g., RF bipolar energy, for output through bipolar electrosurgical plug port 240 and/or RF monopolar energy for output through active monopolar electrosurgical port 250 to surgical instrument 100 to enable one or more electrosurgical functions of surgical instrument 100. With respect to bipolar electrosurgical functions, the RF energy is returned from surgical instrument 100 to surgical generator 200 through bipolar electrosurgical plug port 240 and with respect to monopolar electrosurgical functions, the RF energy is returned to surgical generator 200 via return electrode device 400, e.g., wherein plug 420 of return electrode device 400 is configured to connect to return monopolar electrosurgical plug port 260. It is also contemplated that one or more common ports (not shown) may be configured to act as any two or more of ports 230-260. The electrosurgical and ultrasonic functionalities of surgical generator 200 are described in greater detail below with reference to FIG. 10.

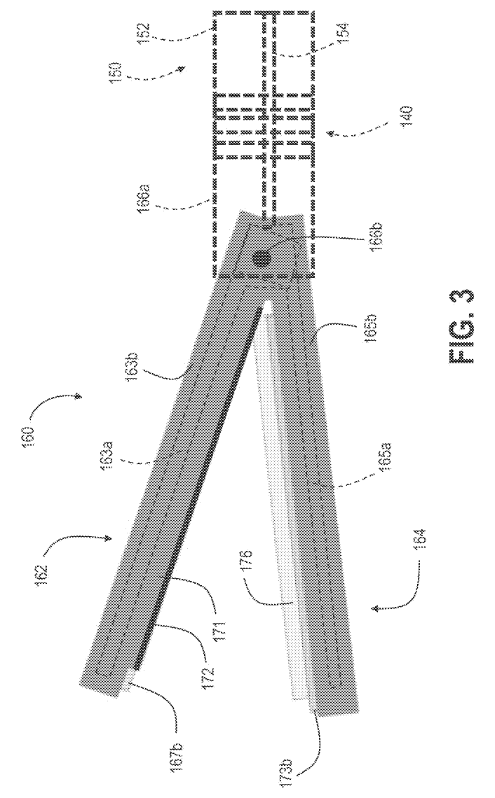

Continuing with reference to FIG. 1, handle assembly 110 of surgical instrument 100 includes a housing 112 defining a body portion and a fixed handle portion. Handle assembly 110 further includes an activation button 120, a movable handle 130, and a drive assembly (not shown). Elongated assembly 150 of surgical instrument 100 extends distally from handle assembly 110 and includes an outer shaft 152, an inner drive 154 (FIG. 3) disposed within outer shaft 152, a rotation knob 156, and end effector assembly 160 including first and second jaw members 162, 164. In aspects, elongated assembly 150 further includes an articulation assembly 140 disposed between and interconnecting outer shaft 152 and end effector assembly 160 with one another to thereby permit articulation of end effector assembly 160 relative to outer shaft 152 (and handle assembly 110) about one or more articulation axes. In aspects, articulation assembly 140 includes a first articulation joint that enables end effector assembly 160 to articulate (e.g., pivot) about a first articulation axis relative to outer shaft 152 (and handle assembly 110) and a second articulation joint that enables end effector assembly 160 to articulate (e.g., pivot) about a second articulation axis relative to outer shaft 152 (and handle assembly 110). In such aspects, the first and second articulation axes may be substantially perpendicular to one another to, for example, enable both yaw and pitch articulation of end effector assembly 160 relative to outer shaft 152 (and handle assembly 110). One or more articulation knobs 142 coupled to the articulation joints of articulation assembly 140 enable operator-controlled articulation of end effector assembly 160 relative to handle assembly 110, e.g., via one or articulation cables (not shown) operably coupled between the one or more articulation knobs 142 and corresponding articulation joints of articulation assembly 140.

Rotation knob 156 is rotatable in either direction to rotate elongated assembly 150 (including end effector assembly 160) in either direction relative to handle assembly 110. Alternatively, rotation knob 156 may rotate end effector assembly 160 independently of outer shaft 152 and articulation assembly 140 (and, thus, relative to outer shaft 152, articulation assembly 140, and handle assembly 110). In either configuration, rotation knob 156 enables roll motion of end effector assembly 160 relative to handle assembly 110.

The drive assembly of handle assembly 110 operably couples a proximal portion of inner drive 154 (FIG. 3) to movable handle 130. The drive assembly may include any suitable components (e.g., linkages, gears, sliders, pivots, motors, springs, etc.) configured to convert the actuation motion of movable handle 130 into movement of inner drive 154 (FIG. 3). Inner drive 154 (FIG. 3) extends from the drive assembly within handle assembly 110 through outer shaft 152 and articulation assembly 140 to end effector assembly 160 wherein a distal portion of inner drive 154 (FIG. 3) is operably coupled to either or both of jaw members 162, 164. As such, movable handle 130 is actuatable relative to housing 112 (e.g., from an un-actuated position farther spaced from housing 112 to an actuated position in closer proximity to the fixed handle portion of housing 112) to thereby actuate the drive assembly to drive inner drive 154 (FIG. 3) to, in turn, move either or both of jaw members 162, 164 relative to the other from a spaced apart position to an approximated position for grasping tissue between jaw members 162, 164. Inner drive 154 (FIG. 3) may include any suitable actuation drive structure(s) such as, for example, drive rod(s), drive cable(s), drive tube(s), drive screw(s), combinations thereof, etc. and may be configured for translational and/or rotational motion to move either or both of jaw members 162, 164 in response to actuation of movable handle 130. End effector assembly 160 is described in greater detail hereinbelow.

In aspects, the drive assembly is configured to provide a jaw grasping force, or jaw grasping force within a jaw grasping force range, to tissue grasped between jaw members 162, 164, e.g., by tuning the drive assembly to provide a desired force or force within a desired range in response to actuation of movable handle 130 and/or by including a force limiting feature whereby the grasping force applied to tissue grasped between jaw members 162, 164 is limited to a particular jaw grasping force or a jaw grasping force within a jaw grasping force range. Additionally or alternatively, the input, e.g., to movable handle 130, may be controlled to thereby regulate the force applied to tissue grasped between jaw members 162, 164.

Referring still to FIG. 1, cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. One or more first electrical lead wires 197 electrically coupled to ultrasonic plug 194 extend through cable 192 and into handle assembly 110 for electrical connection to corresponding electrically conductive components (e.g., circuit traces, lead wires, electrically conductive structures of surgical instrument 100, etc.) of surgical instrument 100 that, in turn, electrical connect to end effector assembly 160 to enable the selective supply of ultrasonic drive signals from surgical generator 200 to end effector assembly 160, e.g., to produce ultrasonic vibration energy for treating tissue, and/or the return of ultrasonic feedback signals from end effector assembly 160 to surgical generator 200, e.g., for ultrasonic sensing. In addition, one or more second electrical lead wires 199 electrically coupled to electrosurgical plug 196 extend through cable 192 and into handle assembly 110 for electrical connection to corresponding electrically conductive components (e.g., circuit traces, lead wires, electrically conductive structures of surgical instrument 100, etc.) of surgical instrument 100 that, in turn, electrical connect to end effector assembly 160 to enable the selective communication of electrosurgical energy between surgical generator 200 and end effector assembly 160. In bipolar configurations, more specifically, at least two separate second electrical lead wires 199 are electrically coupled to end effector assembly 160 such that bipolar electrosurgical energy may be conducted through tissue grasped by or otherwise in contact with end effector assembly 160 to treat the grasped tissue and/or enable electrosurgical sensing. In monopolar configurations, one or more electrical lead wires 199 is electrically coupled to end effector assembly 160 such that monopolar electrosurgical energy may be supplied to tissue from either or both of jaw members 162, 164, e.g., for treating tissue and/or sensing, and return to electrosurgical generator 200 via return electrode device 400.

One or more third electrical lead wires 195 is electrically coupled to activation button 120 such that, in response to a particular activation of activation button 120, surgical generator 200 supplies a corresponding energy or energies to achieve one or more electrosurgical functions and/or one or more ultrasonic functions of end effector assembly 160. Activation button 120 is disposed on housing 112 and, in aspects, may include an ON/OFF switch. In other configurations, activation button 120 may include multiple actuation switches to enable activation from an OFF position to different actuated positions corresponding to different activation settings, e.g., a first actuated position corresponding to a first activation setting and a second actuated position corresponding to a second activation setting. In still other configurations, separate activation buttons may be provided, e.g., a first actuation button for activating a first activation setting and a second activation button for activating a second activation setting. Although two activation settings are detailed, it is understood that additional activation settings may be provided via additional switch positions associated with activation button 120 and/or additional activation button(s). Each activation setting may correspond to one or more electrosurgical functions of end effector assembly 160 and/or one or more ultrasonic functions of end effector assembly 160. Alternatively or additionally, energy activation and/or mode setting may be accomplished at surgical generator 200, e.g., via one or more of user interface features 220.

With reference to FIG. 2, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which an operator (not shown), for example, a surgeon, may be able to telemanipulate robot arms 1002, 1003. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), wherein manual holding and actuation features, e.g., housing 112, actuation button 120, movable handle 130, rotation knob 156, etc. (see FIG. 1), are replaced with robotic inputs. For example, and with momentary reference to FIG. 1, housing 112 may be configured such that mechanical, magnetic, and/or electromechanical engagement features replace the fixed handle portion thereof to enable releasable attachment of housing 112 to one of the attaching devices 1009, 1011. Likewise, movable handle 130, rotation knob 156, and the one or more articulation knobs 142 are replaced with suitable robotic couplers, e.g., rotational input couplers, such that, for example, a motor driven rotational output from the attaching device 1009, 1011 to the corresponding robotic coupler actuates the drive assembly to actuate end effector assembly 160, rotates end effector assembly 160, or articulates end effector assembly 160, respectively. Similarly, rather than providing activation button 120 on housing 112, energy activation may be accomplished via one or more physical or virtual buttons associated with operating console 1005 or other components of robotic surgical system 1000. Further, robotic surgical system 1000 may include or be configured to connect to surgical generator 200, e.g., via cable assembly 190 or in any other suitable manner. In aspects, activation may also be selected (e.g., initiated and/or terminated) from surgical generator 200 (FIG. 1).

Continuing with reference to FIG. 2, the other surgical tool(s) “ST” of robotic surgical system 1000 may include any other suitable surgical instrument(s), e.g., endoscopic cameras, graspers, other surgical tools, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Turning to FIG. 3, end effector assembly 160 is shown disposed at a distal end portion of outer shaft 152 of surgical instrument 100 of surgical system 10 (see FIG. 1), e.g., connected to outer shaft 152 via articulation assembly 140, although end effector assembly 160 may be utilized with any other suitable surgical instrument and/or surgical system. End effector assembly 160 includes a clevis 166a connected to articulation assembly 140 (or to the distal portion of outer shaft 152, in aspects where articulation assembly 140 is omitted or positioned intermediately between proximal and distal portions of outer shaft 152). End effector assembly 160 further includes, as noted above, first and second jaw members 162, 164, at least one of which is movable relative to the other (and clevis 166a), e.g., pivotable about a pivot 166b, between a spaced apart position and an approximated position to grasp tissue between first and second jaw members 162, 164 to enable sealing, dividing, and/or sensing of the grasped tissue, as detailed below.

End effector assembly 160 is shown as a bilateral assembly, e.g., wherein both first and second jaw members 162, 164 are movable relative to one another and clevis 166a (e.g., about pivot 166b which may be a pivot pin or other suitable pivot structure) between the spaced apart position and the approximated position. However, a unilateral assembly, e.g., wherein one jaw member 162, 164 is fixed relative to clevis 166a and the other jaw member 162, 164 is movable relative to the fixed jaw member 162, 164 and clevis 166a between the spaced apart position and the approximated position, is also contemplated. Further, in order to drive movement of either or both of jaw members 162, 164, inner drive 154 of elongated assembly 150 may be coupled to either or both of jaw members 162, 164 via a cam pin-cam slot mechanism, pulley mechanism, linkage mechanism, gear mechanism, lead screw mechanism, or in any other suitable manner such that movement, e.g., translational movement and/or rotational movement, of inner drive 154 moves either or both of jaw members 162, 164 between the spaced apart position and the approximated position, depending upon the direction of movement of inner drive 154. Jaw members 162, 164 may define linear configurations along their lengths, may define curved configurations along their lengths, or may define any other suitable configurations, e.g., including one or more linear, angled, and/or curved sections along their lengths. With respect to at least partially curved or angled configurations, jaw members 162, 164 may be curved or angled in any suitable direction, e.g., wherein jaw members 162, 164 are curved towards and away from one another, respectively, (or vice versa) or wherein jaw members 162, 164 curve laterally (in either direction). Further, jaw members 162, 164 may be formed to include multiple angled sections and/or curves in similar directions, multiple angled sections and/or curves in different directions within a single plane, and/or multiple angled sections and/or curves in different directions in different planes. In addition, jaw members 162, 164 may additionally or alternatively be formed to include other suitable features such as, for example, tapered configuration along their lengths, varying cross-sectional configurations along their lengths, cut outs, indents, edges, protrusions, straight surfaces, curved surfaces, angled surfaces, wide edges, narrow edges, and/or other features.

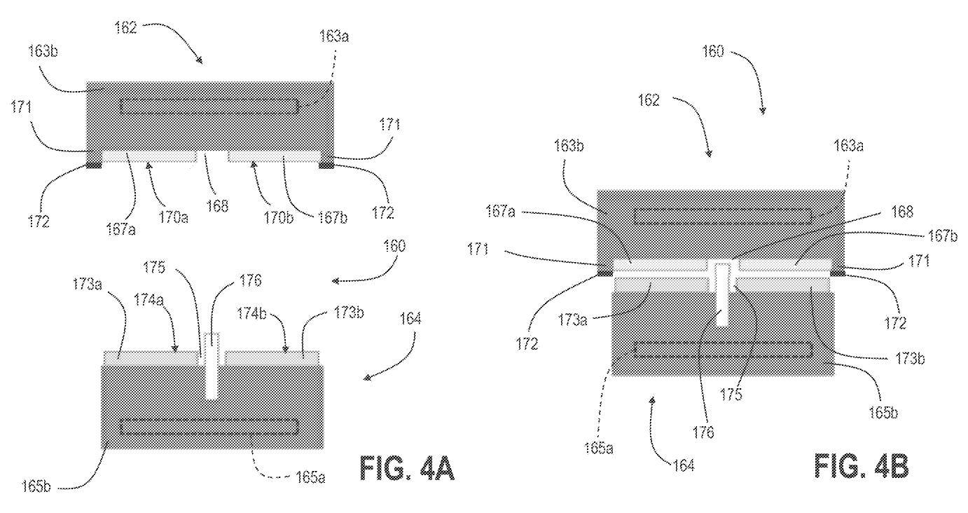

With additional reference to FIGS. 4A and 4B, each jaw member 162, 164 of end effector assembly 160 includes a structural jaw frame 163a, 165a and an insulative jaw body 163b, 165b. Structural jaw frames 163a, 165a provide structural support to jaw members 162, 164, respectively, and may be formed from metal or other suitable material. Insulative jaw bodies 163b, 165b are each formed from one or more components or portions of electrically insulative material(s) and may include, for example, one or more overmolds, spacers, and/or other suitable components. Insulative jaw bodies 163b, 165b are engaged to and may at least partially surround structural jaw frames 163a, 165a.

Jaw member 162 further includes first and second ultrasonic plate portions 167a, 167b disposed on an inner face of insulative jaw body 163b such first and second ultrasonic plate portions 167a, 167b define tissue contacting faces 170a, 170b oriented towards jaw member 164. First and second ultrasonic plate portions 167a, 167b are spaced apart from one another in a transverse direction across jaw member 162 such that a transverse gap 168 is defined between first and second ultrasonic plate portions 167a, 167b along at least portions of the lengths thereof. A portion of insulative jaw body 163b may be exposed within transverse gap 168; in other aspects, jaw member 162 includes a separate component (not shown) disposed within transverse gap 168 such as, for example, a compliant pad, e.g., formed from PTFE, silicone, rubber, or other suitable resiliently compressible (elastomeric) material.

In aspects, first and second ultrasonic plate portions 167a, 167b are spaced apart along the entireties of the lengths thereof and are electrically isolated from one another (and separately electrically coupled to surgical generator 200 (FIG. 1)) to enable independent activation of first and second ultrasonic plate portions 167a, 167b. In other aspects, first and second ultrasonic plate portions 167a, 167b may be joined at the distal ends thereof, e.g., defining a U-shaped configuration, to maintain transverse gap 168 along substantial portions, but not the entireties, of the lengths thereof. In such aspects, first and second ultrasonic plate portions 167a, 167b may share common electrical connections to surgical generator 200 (FIG. 1) (although separate electrical connections are also contemplated).

First and second ultrasonic plate portions 167a, 167b may each include one or more layers of material stacked on top of one another, one or more strips of material positioned side-by-side with one another, and/or one or more concentric rings of material. First and second ultrasonic plate portions 167a, 167b are at least partially formed from or include piezoelectric transducer (PZT) materials and/or other suitable ultrasonic transducer materials (e.g., magnetostrictive materials) such that ultrasonic plate portions 167a, 167b produce mechanical ultrasonic vibration energy in response to receipt of electrical ultrasonic drive signal(s) from surgical generator 200 (FIG. 1) and/or such that ultrasonic plate portions 167a, 167b convert mechanical ultrasonic vibration energy thereof into electrical feedback signal(s) for communication to surgical generator 200 (FIG. 1), thus enabling tissue treatment with ultrasonic energy and/or ultrasonic tissue sensing, respectively.

First and second ultrasonic plate portions 167a, 167b may be configured to produce similar or different vibration patterns. In particular, first and second ultrasonic plate portions 167a, 167b may be configured to produce transverse vibration energy (e.g., laterally in either or both directions across jaw member 162), vibration energy vertically towards jaw member 164, torsional vibration energy, combinations thereof, etc.

In aspects, jaw member 162 includes first and second walls 171 extending along at least a portion of the length of jaw member 162 on the outer peripheral sides of jaw member 162, e.g., laterally outward of first and second ultrasonic plate portions 167a, 167b, respectively. First and second walls 171 extend further towards jaw member 164 as compared to first and second ultrasonic plate portions 167a, 167b. First and second walls 171 may be at least partially formed from or include an electrically conductive material defining electrically conductive surfaces 172 which may be disposed on the laterally inner surfaces of walls 171, the laterally outer surfaces of walls 171, and/or, as shown, on the surfaces of walls 171 oriented towards jaw member 174. Electrically conductive surfaces 172 are electrically isolated from first and second ultrasonic plate portions 167a, 167b, e.g., via insulative jaw body 163b, and are adapted (independently or collectively) to connect to surgical generator 200 (FIG. 1) to enable the conduction of electrosurgical (RF) energy through tissue to treat and/or sense tissue, as detailed below. Walls 171 may further include electrically insulative portions, e.g., on all portions other than surfaces 172. Alternatively, the entireties of walls 171 may be conductive (with or without insulative coatings on portions thereof).

In aspects, walls 171 are entirely electrically insulative and electrically conductive surfaces 172 are omitted. In still other aspects, walls 171 are omitted and electrically conductive surfaces 172 are likewise omitted or otherwise positioned on jaw member 162, e.g., on either or both lateral sides of insulative jaw body 163b and/or on an outer face of insulative jaw body 163b. In aspects where provided, walls 171 and/or electrically conductive surfaces 172 may terminate proximally of the distal extents of first and second ultrasonic plate portions 167a, 167b (as shown in FIG. 3).

Continuing with reference to FIGS. 3 and 4A-4B, jaw member 164 further includes first and second electrically conductive plate portions 173a, 173b disposed on an inner face of insulative jaw body 165b such that first and second electrically conductive plate portions 173a, 173b define tissue contacting faces 174a, 174b oriented towards jaw member 162. As such, in the approximated position of jaw members 162, 164, tissue is grasped between tissue contacting faces 174a, 174b of first and second electrically conductive plate portions 173a, 173b of jaw member 164 and tissue contacting faces 170a, 170b of first and second ultrasonic plate portions 167a, 167b of jaw member 162, respectively.

First and second electrically conductive plate portions 173a, 173b are spaced apart from one another in a transverse direction across jaw member 164 such that a transverse gap 175 is defined between electrically conductive plate portions 173a, 173b along at least portions of the lengths thereof. The term “plate” as utilized herein is not limited to any particular method of formation or resultant thickness; indeed, the term “plate” as utilized herein includes pre-formed and subsequently attached “plates,” “plates” directly deposited onto another component (e.g., via spraying, deposition techniques, etc.), “plates” formed from removal of material to expose an underlying layer, etc.

In aspects, first and second electrically conductive plates portions 173a, 173b are spaced apart along the entireties of the lengths thereof and are electrically isolated from one another (and separately electrically coupled to surgical generator 200 (FIG. 1)) to enable independent activation of first and second electrically conductive plates portions 173a, 173b. In other aspects, first and second electrically conductive plates portions 173a, 173b may be joined at the distal ends thereof, e.g., defining a U-shaped configuration, to maintain transverse gap 175 along substantial portions, but not the entireties, of the lengths thereof. In such aspects, first and second electrically conductive plates portions 173a, 173b may share common electrical connections to surgical generator 200 (FIG. 1) (although separate electrical connections are also contemplated).

Electrically conductive plates portions 173a, 173b are adapted to connect to surgical generator 200 (FIG. 1) to enable energization of electrically conductive plates portions 173a, 173b with RF energy. In aspects, electrically conductive plates portions 173a, 173b are electrically isolated and energizable with RF energy at different potentials for conducting electrosurgical energy between electrically conductive plates portions 173a, 173b and through tissue grasped between jaw members 162, 164 to treat, e.g., seal, the grasped tissue. In such configurations, RF energy flows substantially transversely, e.g., laterally across jaw member 164 and through the grasped tissue, to treat the tissue. Alternatively or additionally, electrically conductive plates portions 173a, 173b are energizable with RF energy at different potentials compared to either or both of electrically conductive surfaces 172 of walls 171 of jaw member 162 (in aspects where electrically conductive surfaces 172 are provided) such that electrosurgical energy is conducted between jaw members 162, 164 and through tissue grasped therebetween to treat, e.g., seal, the grasped tissue. The above-noted electrical paths, although described with respect to tissue treatment, likewise apply to electrosurgical tissue sensing.

Referring still to FIGS. 3 and 4A-4B, a blade 176 engaged within insulative jaw body 165b of jaw member 164 is disposed within transverse gap 175 and protrudes from transverse gap 175 beyond electrically conductive plate portions 173a, 173b of jaw member 164 farther towards jaw member 162 such that, in the approximated position of jaw members 162, 164 (FIG. 4B), blade 176 is disposed in close proximity with or extends into transverse gap 168 of jaw member 162. In other aspects, this configuration is reversed, e.g., wherein blade 176 is disposed on jaw member 162 and extends to or into transverse gap 175 of jaw member 164 in the approximated position of jaw members 162, 164 (FIG. 4B). Blade 176 may define a bunt tissue contacting face, one or more tissue contacting edges, and/or any other suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 176 is at least partially formed from or includes piezoelectric transducer (PZT) materials and/or other suitable ultrasonic transducer materials (e.g., magnetostrictive materials) and is electrically connected to surgical generator 200 (FIG. 1) such that blade 176 produces mechanical ultrasonic vibration energy in response to receipt of electrical ultrasonic drive signal(s) from surgical generator 200 (FIG. 1) and/or such that blade 176 converts mechanical ultrasonic vibration energy thereof into electrical feedback signal(s) for communication to surgical generator 200 (FIG. 1), thus enabling tissue treatment with ultrasonic energy, e.g., tissue dissection, and/or ultrasonic tissue sensing, respectively. Blade 176 may be configured to produce any suitable vibration pattern(s) such as, for example, transverse vibration energy (e.g., laterally in either or both directions across jaw member 164), vibration energy vertically towards jaw member 162, torsional vibration energy, combinations thereof, etc.

Blade 176 and/or ultrasonic plate portions 167a, 167b may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; Electro Bond® coating available from Surface Solutions Group of Chicago, IL, USA; or other suitable coatings and/or methods of applying coatings.

With continued reference to FIGS. 3 and 4A-4B, in use, jaw members 162, 164 are moved from the spaced apart position (FIG. 4A) to the approximated position (FIG. 4B) to grasp tissue therebetween and, more specifically, between tissue contacting faces 170a, 170b of first and second ultrasonic plate portions 167a, 167b of jaw member 162, and tissue contacting faces 174a, 174b of first and second electrically conductive plate portions 173a, 173b of jaw member 164, respectively. In aspects, in the approximated position (FIG. 4B), walls 171 at least partially overlap jaw member 164 on the outer lateral sides thereof. Alternatively, walls 171 may extend to tissue contacting faces 174a, 174b or may be vertically spaced from tissue contacting faces 174a, 174b.

With tissue grasped between jaw members 162, 164, first and second electrically conductive plate portions 173a, 173b and/or electrically conductive surfaces 172 are energized with RF energy at two or more different potentials to establish at least one potential gradient for the conduction of RF energy therebetween and through the grasped tissue. As noted above, RF energy (e.g., current) may flow transversely across jaw members 162, 164 and/or between jaw members 162, 164 and through the tissue grasped therebetween. The conduction of RF energy through the tissue (transversely and/or vertically between jaw members 162, 164) heats the grasped tissue (via joule heating) to, for example, seal or otherwise treat the grasped tissue. In aspects, surgical generator 200 (FIG. 1) is configured to implement current-based control, voltage-based control, tissue impedance-based control, duration-based control, combinations thereof, and/or other suitable control of the RF energy to achieve the desired tissue treatment, e.g., tissue sealing.

Simultaneously, overlapping, alternating, or in any other suitable manner with the supply of RF energy, an ultrasonic drive signal is supplied to ultrasonic plate portions 167a, 167b to cause ultrasonic plate portions 167a, 167b to ultrasonically vibrate against the grasped tissue. The transmission of this ultrasonic vibration energy to the grasped tissue heats the grasped tissue (via conductive heating), thus facilitating tissue sealing (or other tissue treatment). Surgical generator 200 (FIG. 1) may control the ultrasonic drive signal to achieve a desired ultrasonic vibrational output of ultrasonic plate portions 167a, 167b and may vary the ultrasonic drive signal to achieve different ultrasonic vibrational outputs, e.g., a first output corresponding to a first magnitude of vibration (e.g., LOW power) and a second output corresponding to a second, greater magnitude of vibration (e.g., HIGH power). Thus, both the RF energy and ultrasonic energy may be controlled to facilitate tissue sealing or other tissue treatment.

With respect to tissue sensing, surgical generator 200 (FIG. 1) may monitor the impedance of the grasped tissue by monitoring the RF signals transmitted to and from jaw members 162, 164. Surgical generator 200 (FIG. 1) may also receive feedback from ultrasonic plate portions 167a, 167b via the electrical connections thereto to enable ultrasonic plate portions 167a, 167b to function as ultrasonic sensors capable of sensing tissue temperature, tissue stiffness, and/or jaw pressure on tissue. With these RF and/or ultrasonic sensed parameters, surgical generator 200 (FIG. 1) can control either or both energy modalities to achieve and maintain a desired tissue temperature and/or tissue temperature curve. Surgical generator 200 (FIG. 1) may additionally or alternatively, using these sensed parameters, evaluate the status of tissue sealing and/or determine when tissue sealing is complete, e.g., based upon whether the sensed parameters meet pre-determined criteria.

Once the grasped tissue is sealed, or otherwise where it is desired to dissect the grasped tissue, blade 176 may be activated by an ultrasonic drive signal to ultrasonically energize blade 176 to heat and ultimately dissect the sealed tissue across the tissue seal. The geometry of blade 176 facilitates tissue dissection while the geometries of ultrasonic plate portions 167a, 167b facilitate tissue sealing. Electrically conductive plate portions 173a, 173b and/or ultrasonic plate portions 167a, 167b may be turned off (e.g., unenergized) during tissue dissection; alternatively, electrically conductive plate portions 173a, 173b and/or ultrasonic plate portions 167a, 167b may be energized for tissue treatment and/or tissue sensing during energization of blade 176 to facilitate tissue dissection. Further, in aspects, blade 176 may be utilized for ultrasonic sensing (similarly as detailed above) during tissue sealing and/or tissue dissection.

Blade 176 may additionally or alternatively be used for open jaw dissection, e.g., by moving end effector assembly 160 relative to tissue to enable blade 176 to dynamically cut the tissue as end effector assembly 160 is moved relative to the tissue.

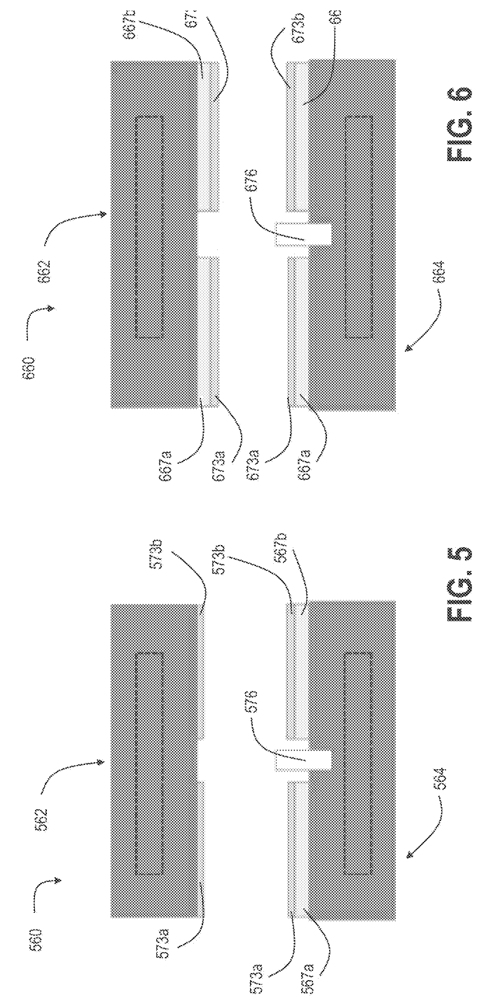

Turning to FIG. 5, another end effector assembly 560 provided in accordance with the present disclosure is shown. End effector assembly 560 is similar to and may include any of the features of end effector assembly 160 (FIGS. 3-4B). Accordingly, for purposes of brevity, only the differences between end effector assembly 560 and end effector assembly 160 (FIGS. 3-4B) are described in detail below while similarities are summarily described or omitted entirely.

End effector assembly 560 includes ultrasonic plate portions 567a, 567b disposed on one of the jaw members 562, 564, e.g., jaw member 564, and electrically conductive plate portions 573a, 573b disposed on both jaw members 562, 564. With respect to jaw member 564, which includes both electrically conductive plate portions 573a, 573b and ultrasonic plate portions 567a, 567b, electrically conductive plate portions 573a, 573b may be stacked on ultrasonic plate portions 567a, 567b, respectively, although the opposite configuration is also contemplated. Electrically conductive plate portions 573a, 573b of jaw members 562, 564 may be charged to different potentials in any suitable manner and, in aspects, may be configurable by surgical generator 200 (FIG. 1). For example, in aspects, electrically conductive plate portions 573a, 573b of jaw member 562 may be charged to a first potential and electrically conductive plate portions 573a, 573b of jaw member 564 charged to a second, different potential for conducting energy vertically between jaw members 562, 564. In other aspects, electrically conductive plate portions 573a of jaw members 562, 564 may be charged to a first potential and electrically conductive plate portions 573b of jaw members 562, 564 charged to a second, different potential for conducting energy transversely across jaw members 562, 564.

Continuing with reference to FIG. 5, blade 576 is shown engaged with jaw member 564 although blade 576 may alternatively be engaged with jaw member 562. End effector assembly 560 may be utilized similarly as detailed above for electrosurgical and/or ultrasonic tissue treatment and/or sensing and also for ultrasonic tissue dissection (with electrosurgical and/or further ultrasonic assistance, in aspects).

With reference to FIG. 6, end effector assembly 660 is similar to and may include any of the features of end effector assembly 560 (FIG. 5) except that both jaw members 662, 664 include electrically conductive plate portions 673a, 673b stacked on ultrasonic plate portions 667a, 667b, respectively, although the opposite configuration is also contemplated. End effector assembly 660 may define any of the electrical paths detailed above with respect to end effector assembly 560 (FIG. 5) and/or be utilized similarly as detailed above for electrosurgical and/or ultrasonic tissue treatment and/or sensing and also for ultrasonic tissue dissection (with electrosurgical and/or further ultrasonic assistance, in aspects).

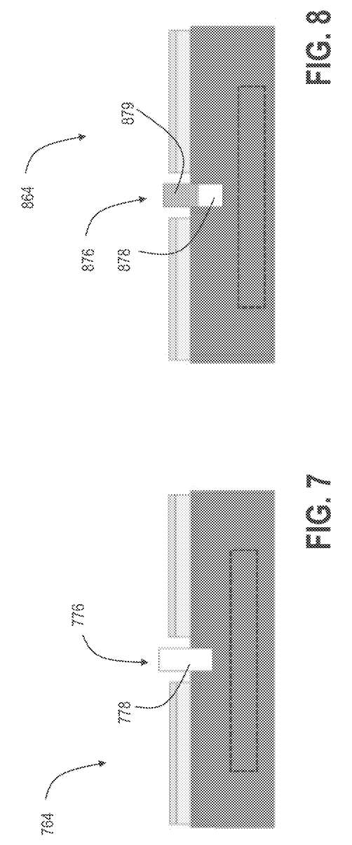

Referring to FIGS. 7 and 8, different configurations of blades 776, 876, respectively, provided in accordance with the present disclosure are shown. Blades 776, 876 are shown engaged with jaw members 764, 864, respectively, although blades 776, 876 may be utilized with any of the jaw members detailed herein or any other suitable jaw members. Blade 776, as shown in FIG. 7, is formed from a PZT crystal 778 that, when energized with an ultrasonic drive signal, is driven to produce mechanical ultrasonic vibration energy. The PZT crystal 778, in turn, is configured to transmit the mechanical ultrasonic vibration energy to tissue in contact therewith. Blade 876, on the other hand, as shown in FIG. 8, includes a PZT crystal base 878 and a blade body 879 attached to PZT crystal base 878, e.g., stacked on, receiving within, surrounding, or otherwise attached to PZT crystal base 878. PZT crystal base 878 is configured to be energized with an ultrasonic drive signal to produce mechanical ultrasonic vibration energy that is transmitted to blade body 879. Blade body 879, in turn, is configured to transmit the mechanical ultrasonic vibration energy to tissue in contact therewith. In aspects, blade body 879 is formed from ceramic, titanium (or other suitable metal), or other suitable material.

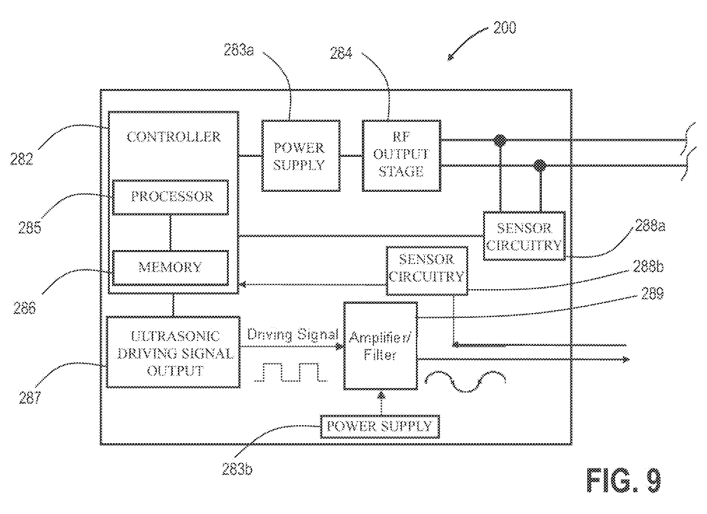

FIG. 9 illustrates surgical generator 200 and, more specifically, schematically illustrates the internal operable features associated with the electrosurgical and ultrasonic functionality thereof. Surgical generator 200 includes a controller 282, one or more power supplies 283a, 283b, an RF output stage 284, RF sensor circuitry 288a, an ultrasonic driving signal output 287, and amplifier/filter 289, and ultrasonic sensor circuitry 288b. Controller 282 includes one or more processors 285 and associated memory(s) 286, e.g., storing instructions to be executed by processor(s) 285 to control the electrosurgical energy output by RF output stage 284, control the ultrasonic driving signal output by ultrasonic driving signal output 287, receive the electrosurgical feedback from RF sensor circuitry 288a, and receive the ultrasonic feedback from ultrasonic sensor circuitry 288b.

Power supply 283a may be a high voltage DC power supply configured to provide high voltage DC power to RF output stage 284 which converts the high voltage DC power into RF electrosurgical energy for delivery to end effector assembly 160 (FIGS. 3-4B), e.g., to one or more of the electrosurgical components of end effector assembly 160: electrically conductive plate portions 173a, 173b and/or electrically conductive surfaces 172 (see FIGS. 3-4B); and to receive return energy from one or more of the electrosurgical components of end effector assembly 160 (FIGS. 3-4B) to complete the circuit back to surgical generator 200. RF sensor circuitry 288a is operably coupled to the input and output electrical lines to and from RF output stage 284 to sense electrical parameters of the energy delivered to and returned from end effector assembly 160 (FIGS. 3-4B), e.g., voltage, current, resistance, etc. thereof, and, based thereon, determine one or more parameters of tissue, e.g., impedance of tissue. RF sensor circuitry 288a provides feedback, e.g., based on the sensed parameter(s) of tissue, to controller 282 which, in turn, selects an energy-delivery algorithm, modifies an energy-delivery algorithm, and/or adjust energy-delivery parameters for electrosurgical and/or ultrasonic energy based at least in part on the sensed parameter(s) of tissue.

In addition or as an alternative to controller 282 controlling the supply of electrosurgical and/or ultrasonic energy to treat tissue, controller 282 may also be configured to interrogate (e.g., sense) tissue such as, for example, where RF sensor circuitry 288a senses one or more electrical parameters to provide feedback to controller 282 such as, for example, to enable determination of the impedance of tissue. That is, instead of or together with electrosurgical tissue treatment, surgical generator 200 enables interrogating tissue with electrosurgical energy (without the need to supply tissue-treating electrosurgical energy as detailed above). Tissue interrogation may be initiated in a bipolar electrosurgical interrogation mode and/or a monopolar electrosurgical interrogation mode. In aspects, electrosurgical interrogating may be performed in the absence of any treatment energy, e.g., with the electrosurgical and ultrasonic treatment energy turned off, e.g., to assess tissue before treatment (e.g., to determine a type of treatment, a suitable energy delivery algorithm, and/or suitable energy delivery parameter), after treatment (e.g., to determine completion of tissue treatment and/or a state of treated or surrounding tissue), or in other circumstances.

With respect to interrogation of tissue, controller 282 is configured to transmit an interrogation signal according to any of the electrosurgical paths detailed above such that the signal is returned to controller 282 to enable evaluation thereof. The interrogation signal may be a continuous signal, a pulse signal, or a plurality of pulses. Controller 282, more specifically, is configured to evaluate the returned signal, e.g., the voltage, current, resistance, etc. thereof, and, based thereon, determine one or more parameters of tissue, e.g., the impedance of tissue, which is indicative of whether tissue is sufficiently sealed. Controller 282 may then, for example, select an energy-delivery algorithm, modify an energy-delivery algorithm, and/or adjust energy-delivery parameters based thereon, including stopping the delivery of energy.

Continuing with reference to FIG. 9, power supply 283b (which may be the same as or separate from power supply 283a), ultrasonic driving signal output 287, and amplifier/filter 289 are configured to generate an ultrasonic driving signal that is output to one or more of the ultrasonic components of end effector assembly 160: ultrasonic plate portions 167a, 167b and/or blade 176 (see FIGS. 3-4B) for treating, e.g., sealing and/or dissecting, tissue. Further, ultrasonic sensor circuitry 288b is configured to monitor an electrical input from one or more of the ultrasonic components of end effector assembly 160, e.g., voltage, current, resistance, etc. to provide ultrasonic sensing, e.g., to determine one or more parameters of tissue such as, for example, tissue temperature, tissue stiffness, and/or jaw pressure on tissue. These sensed parameter(s) of tissue are communicated to controller 282 which, in turn, selects an energy-delivery algorithm, modifies an energy-delivery algorithm, and/or adjust energy-delivery parameters (including stopping the delivery of energy) for electrosurgical and/or ultrasonic energy based at least in part on the sensed parameter(s) of tissue.

As demonstrated above, the end effector assemblies and surgical generators of the present disclosure enable RF energy tissue treatment for sealing tissue and/or for facilitating dissection of tissue, ultrasonic energy tissue treatment to facilitate sealing tissue and/or for dissecting tissue, RF tissue sensing (e.g., for sensing tissue impedance), and ultrasonic tissue sensing (e.g., for sensing tissue temperature, tissue stiffness, and/or jaw pressure on tissue). Some or all of this sensed feedback may be utilized to modulate the application of electrosurgical and/or ultrasonic energy to efficiently and effectively treat, e.g., seal and/or dissect, tissue.

Aspects of this disclosure may be further described by reference to the following numbered paragraphs:

1. A surgical instrument (100), comprising: an end effector assembly (160, 560, 660), including: first and second jaw members (162, 164; 562, 564; 662, 664), at least one of the first jaw member or the second jaw member movable relative to the other between a spaced apart position and an approximated position for grasping tissue therebetween, the first jaw member including first and second electrically conductive tissue contacting plate portions (173a, 173b; 573a, 573b; 673a, 673b) configured to conduct Radio Frequency (RF) energy through tissue grasped between the first and second jaw members, the second jaw member including first and second ultrasonic tissue contacting plate portions (167a, 167b; 567a, 567b; 667a, 667b) configured to produce ultrasonic vibration energy and transmit the ultrasonic vibration energy to tissue grasped between the first and second jaw members.

2. The surgical instrument (100) according to paragraph 1, wherein one of the first jaw member or the second jaw member further includes first and second walls (171) extending from respective first and second outer peripheral sides thereof, the first and second walls (171) including first and second electrically conductive surfaces (172), respectively, the first and second electrically conductive surfaces (172) electrically isolated from the first and second electrically conductive tissue contacting plate portions (173a, 173b; 573a, 573b; 673a, 673b) and configured to be energized to different potentials compared to the first and second electrically conductive tissue contacting plate portions to conduct RF energy therebetween and through tissue grasped between the first and second jaw members.

3. The surgical instrument (100) according to paragraph 1 or 2, wherein the second jaw member further includes third and fourth electrically conductive tissue contacting plate portions (573a, 573b; 673a, 673b), at least two of the first, second, third, or fourth electrically conductive tissue contacting plate portions electrically isolated and configured to be energized to different potentials to conduct RF energy therebetween and through tissue grasped between the first and second jaw members.

4. The surgical instrument (100) according to paragraph 3, wherein the third electrically conductive tissue contacting plate portion, and the first ultrasonic tissue contacting plate portion are stacked relative to one another on the second jaw member, and wherein the fourth electrically conductive tissue contacting plate portion and the second ultrasonic tissue contacting plate portion are stacked relative to one another on the second jaw member.

5. The surgical instrument (100) according to any preceding paragraph, wherein the first jaw member further includes third and fourth ultrasonic tissue contacting plate portions (667a, 667b) configured to produce ultrasonic vibration energy and transmit the ultrasonic vibration energy to tissue grasped between the first and second jaw members.

6. The surgical instrument (100) according to paragraph 5, wherein the first electrically conductive tissue contacting plate portion and the third ultrasonic tissue contacting plate portion are stacked relative to one another on the first jaw member, and wherein the second electrically conductive tissue contacting plate portion and the fourth ultrasonic tissue contacting plate portion are stacked relative to one another on the first jaw member.

7. The surgical instrument (100) according to any preceding paragraph, wherein the first and second ultrasonic tissue contacting plate portions are configured to sense at least one parameter of tissue in contact therewith.

8. The surgical instrument (100) according to any preceding paragraph, wherein one of the first jaw member or the second jaw member includes an ultrasonic blade (176, 576, 676, 776, 876) disposed between the plate portions thereof, the ultrasonic blade configured to produce ultrasonic vibration energy and transmit the ultrasonic vibration energy to tissue grasped between the first and second jaw members.

9. The surgical instrument (100) according to paragraph 8, wherein the ultrasonic blade is formed from a piezoelectric material (778) or includes a piezoelectric base (878) and a transmission body (879) coupled to the piezoelectric base.

10. The surgical instrument (100) according to paragraph 8 or 9, wherein the ultrasonic blade is configured to sense at least one parameter of tissue in contact therewith.

11. The surgical instrument (100) according to any preceding paragraph, wherein the first and second electrically conductive tissue contacting plate portions are connected at ends thereof to define a U-shaped configuration or wherein the first and second electrically conductive tissue contacting plate portions are electrically isolated from one another.

12. The surgical instrument (100) according to any preceding paragraph, further comprising a surgical robot (1002, 1003) wherein the end effector assembly (160) is configured to releasably connect to the surgical robot or further comprising a handle assembly (110) wherein the end effector assembly (160) is operably coupled to the handle assembly.

13. A surgical system, comprising: an end effector assembly (160, 560, 660) including first and second jaw members (162, 164; 562, 564; 662, 664), at least one of the first jaw member or the second jaw member movable relative to the other between a spaced apart position and an approximated position for grasping tissue therebetween, the end effector assembly further including at least one electrically conductive tissue contacting plate portion (173a, 173b; 573a, 573b; 673a, 673b) and at least one ultrasonic tissue contacting plate portion (167a, 167b; 567a, 567b; 667a, 667b); and a surgical generator (200) configured to: output Radio Frequency (RF) treatment energy to the at least one electrically conductive tissue contacting plate portion, output an ultrasonic drive signal to the at least one ultrasonic tissue contacting plate portion, sense feedback from the at least one electrically conductive tissue contacting plate portion, and sense feedback from the at least one ultrasonic tissue contacting plate portion, the surgical generator configured to control the output of the RF treatment energy and the output of the ultrasonic drive signal to seal tissue grasped between the first and second jaw members based on the sensed feedback from the at least one electrically conductive tissue contacting plate portion and the sensed feedback from the at least one ultrasonic tissue contacting plate portion.

14. The surgical system according to paragraph 13, wherein the surgical generator is configured to determine at least one of: a temperature of tissue grasped between the first and second jaw members, a stiffness of tissue grasped between the first and second jaw members, or a jaw pressure applied to tissue grasped between the first and second jaw members based on the sensed feedback from the at least one ultrasonic tissue contacting plate portion.

15. The surgical system according to paragraph 13 or 14, wherein the surgical generator is configured to determine an impedance of tissue grasped between the first and second jaw members based on the sensed feedback from the at least one electrically conductive tissue contacting plate portion.

16. The surgical system according to any one of paragraphs 13-15, wherein the end effector assembly further includes an ultrasonic blade (176, 576, 676, 776, 876) engaged to one of the first jaw member or the second jaw members, and wherein the surgical generator is further configured to output an ultrasonic drive signal to the ultrasonic blade to dissect tissue grasped between the first and second jaw members.

17. The surgical system according to any one of paragraphs 13-16, wherein the surgical generator is configured to control the output using temperature-based control and/or wherein the surgical generator is configured to control the output to terminate the delivery of energy when the sensed feedback indicates that tissue grasped between the first and second jaw members is sealed.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
an end effector assembly, including:
first and second jaw members, at least one of the first jaw member or the second jaw member movable relative to the other between a spaced apart position and an approximated position for grasping tissue therebetween,
the first jaw member including first and second electrically conductive tissue contacting plate portions configured to conduct Radio Frequency (RF) energy through tissue grasped between the first and second jaw members,
the second jaw member including first and second ultrasonic tissue contacting plate portions configured to produce ultrasonic vibration energy and transmit the ultrasonic vibration energy to tissue grasped between the first and second jaw members.

2. The surgical instrument according to claim 1, wherein one of the first jaw member or the second jaw member further includes first and second walls extending from respective first and second outer peripheral sides thereof, the first and second walls including first and second electrically conductive surfaces, respectively, the first and second electrically conductive surfaces electrically isolated from the first and second electrically conductive tissue contacting plate portions and configured to be energized to different potentials compared to the first and second electrically conductive tissue contacting plate portions to conduct RF energy therebetween and through tissue grasped between the first and second jaw members.

3. The surgical instrument according to claim 1, wherein the second jaw member further includes third and fourth electrically conductive tissue contacting plate portions, at least two of the first, second, third, or fourth electrically conductive tissue contacting plate portions electrically isolated and configured to be energized to different potentials to conduct RF energy therebetween and through tissue grasped between the first and second jaw members.

4. The surgical instrument according to claim 3, wherein the third electrically conductive tissue contacting plate portion and the first ultrasonic tissue contacting plate portion are stacked relative to one another on the second jaw member, and wherein the fourth electrically conductive tissue contacting plate portion and the second ultrasonic tissue contacting plate portion are stacked relative to one another on the second jaw member.

5. The surgical instrument according to claim 1, wherein the first jaw member further includes third and fourth ultrasonic tissue contacting plate portions configured to produce ultrasonic vibration energy and transmit the ultrasonic vibration energy to tissue grasped between the first and second jaw members.

6. The surgical instrument according to claim 5, wherein the first electrically conductive tissue contacting plate portion and the third ultrasonic tissue contacting plate portion are stacked relative to one another on the first jaw member, and wherein the second electrically conductive tissue contacting plate portion and the fourth ultrasonic tissue contacting plate portion are stacked relative to one another on the first jaw member.

7. The surgical instrument according to claim 1, wherein the first and second ultrasonic tissue contacting plate portions are configured to sense at least one parameter of tissue in contact therewith.

8. The surgical instrument according to claim 1, wherein one of the first jaw member or the second jaw member includes an ultrasonic blade disposed between the plate portions thereof, the ultrasonic blade configured to produce ultrasonic vibration energy and transmit the ultrasonic vibration energy to tissue grasped between the first and second jaw members.

9. The surgical instrument according to claim 8, wherein the ultrasonic blade is formed from a piezoelectric material.

10. The surgical instrument according to claim 8, wherein the ultrasonic blade includes a piezoelectric base and a transmission body coupled to the piezoelectric base.

11. The surgical instrument according to claim 8, wherein the ultrasonic blade is configured to sense at least one parameter of tissue in contact therewith.

12. The surgical instrument according to claim 1, wherein the second jaw member further includes a structural jaw frame supporting an insulative jaw body thereon, and wherein the first and second ultrasonic tissue contacting plate portions are disposed on the insulative jaw body in spaced relation relative to one another.

13. The surgical instrument according to claim 1, further comprising a surgical robot, wherein the end effector assembly is configured to releasably connect to the surgical robot.

14. The surgical instrument according to claim 1, further comprising a handle assembly, wherein the end effector assembly is operably coupled to the handle assembly.

15. A surgical system, comprising:
an end effector assembly including first and second jaw members, at least one of the first jaw member or the second jaw member movable relative to the other between a spaced apart position and an approximated position for grasping tissue therebetween, the end effector assembly further including at least one electrically conductive tissue contacting plate portion and at least one ultrasonic tissue contacting plate portion; and
a surgical generator configured to: output Radio Frequency (RF) treatment energy to the at least one electrically conductive tissue contacting plate portion, output an ultrasonic drive signal to the at least one ultrasonic tissue contacting plate portion, sense feedback from the at least one electrically conductive tissue contacting plate portion, and sense feedback from the at least one ultrasonic tissue contacting plate portion, the surgical generator configured to control the output of the RF treatment energy and the output of the ultrasonic drive signal to seal tissue grasped between the first and second jaw members based on the sensed feedback from the at least one electrically conductive tissue contacting plate portion and the sensed feedback from the at least one ultrasonic tissue contacting plate portion.

16. The surgical system according to claim 15, wherein the surgical generator is configured to determine at least one of: a temperature of tissue grasped between the first and second jaw members, a stiffness of tissue grasped between the first and second jaw members, or a jaw pressure applied to tissue grasped between the first and second jaw members based on the sensed feedback from the at least one ultrasonic tissue contacting plate portion.

17. The surgical system according to claim 16, wherein the surgical generator is configured to determine an impedance of tissue grasped between the first and second jaw members based on the sensed feedback from the at least one electrically conductive tissue contacting plate portion.

18. The surgical system according to claim 15, wherein the end effector assembly further includes an ultrasonic blade engaged to one of the first jaw member or the second jaw members, and wherein the surgical generator is further configured to output an ultrasonic drive signal to the ultrasonic blade to dissect tissue grasped between the first and second jaw members.

19. The surgical system according to claim 15, wherein the surgical generator is configured to control the output using temperature-based control.

20. The surgical system according to claim 15, wherein the surgical generator is configured to control the output to terminate the delivery of energy when the sensed feedback indicates that tissue grasped between the first and second jaw members is sealed.
